(19) United States
(12) Patent Application Publication
(10) Pub. No.: US 2026/0256338 A1
(43) Pub. Date: Sep. 3, 2026

(54) SYSTEMS AND METHODS FOR DETECTING AND MITIGATING EXTRANEOUS LIGHT AT A SCENE

(71) Applicant: Intuitive Surgical Operations, Inc., Sunnyvale, CA (US)

(72) Inventors: Ramin Samadani, Menlo Park, CA (US); Jeffrey M. DiCarlo, Austin, TX (US); William Jason Culman, Scotts Valley, CA (US)

(21) Appl. No.: 18/880,151

(22) PCT Filed: Aug. 11, 2023
(86) PCT No.: PCT/US2023/030084

§ 371 Date: Dec. 30, 2024

Related U.S. Application Data
(60) Provisional application No. 63/397,969, filed on Aug. 15, 2022.

(51) Int. Cl. A61B 1/00; A61B 1/04; A61B 1/07

(52) U.S. Cl. CPC A61B 1/000094; A61B 1/000095; A61B 1/043; A61B 1/07

(57) ABSTRACT

An extraneous light detection system may obtain first light images of a scene illuminated with first light in a first waveband and captured over a time period. The first light images depict a subject located at the scene. The extraneous light detection system may track, in the first light images over the time period, pixel values of a target region of the subject. The extraneous light detection system may determine, based on a comparison of signal levels of pixels that depict the target region of the subject with a background model representative of reflectivity of the target region, whether the target region is illuminated with extraneous first light.

### BACKGROUND INFORMATION

The present application claims priority to U.S. Provisional Patent Application No. 63/397,969, filed Aug. 15, 2022, the contents of which is hereby incorporated by reference in its entirety.

### BACKGROUND INFORMATION

During a minimally-invasive surgical procedure, an imaging system (e.g., an endoscope) may be used to capture images of a scene illuminated with light from a light source. The images (e.g., a video stream) may be presented during the surgical procedure to assist the surgeon in performing the surgical procedure. In some scenarios, the images of the scene are or are augmented with fluorescence images. Fluorescence images are generated based on detected fluorescence emitted by fluorophores when the fluorophores are excited by fluorescence excitation light (e.g., near-infrared (NIR) light). The fluorescence images may be used, for example, to highlight certain portions of the scene, certain types of tissue, or tissue perfusion of the surgical area in a selected color (e.g., green).

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system may comprise a memory storing instructions and a processor communicatively coupled to the memory and configured to execute the instructions to: obtain first light images of a scene illuminated with first light in a first waveband and captured over a time period, the first light images depicting a subject located at the scene; track, in the first light images over the time period, pixel values of a target region of the subject; and determine, based on a comparison of signal levels of pixels that depict the target region of the subject with a background model representative of reflectivity of the target region, whether the target region is illuminated with extraneous first light.

An illustrative method may comprise obtaining first light images of a scene illuminated with first light in a first waveband and captured over a time period, the first light images depicting a subject located at the scene; tracking, in the first light images over the time period, pixel values of a target region of the subject; and determining, based on a comparison of signal levels of pixels that depict the target region with a background model representative of reflectivity of the target region, whether the target region is illuminated with extraneous first light.

An illustrative system may comprise a memory storing instructions and a processor communicatively coupled to the memory and configured to execute the instructions to: obtain visible light images of a scene illuminated with visible light and fluorescence excitation light and captured over a time period, the visible light images depicting a subject located at the scene; track, in the visible light images over the time period, pixel values of a target region of the subject; adjust signal levels of pixels, in the visible light images, that depict the target region based on an incident visible light model that estimates an amount of incident visible light from a light source of the scene at each pixel as a function of pixel position and depth of the target region representative of a three-dimensional spatial distribution of visible light from a light source; determine, based on a comparison of the adjusted signal levels of the pixels that depict the target region with a background model that models signal levels of the pixels that depict the target region illuminated with ideal light, that the target region is illuminated with extraneous fluorescence excitation light; and perform, based on the determination that the target region is illuminated with extraneous fluorescence excitation light, an extraneous light mitigation operation.

An illustrative system may comprise a memory storing instructions and a processor communicatively coupled to the memory and configured to execute the instructions to: obtain an image of a scene illuminated with light, the image depicting a subject at the scene; determine, based on the image and a background model representative of reflectivity of a target region of the subject, that the target region is illuminated with extraneous light; and perform, based on the determination that the target region is illuminated with extraneous light, an extraneous light mitigation operation.

An illustrative system may comprise a memory storing instructions and a processor communicatively coupled to the memory and configured to execute the instructions to: obtain a first light image of a scene illuminated with first light and a second light image of the scene illuminated with second light; determine, based on the first light image and a background model representative of reflectivity of a target region of a subject at the scene, that the target region is illuminated with extraneous second light; and adjust, based on the determination that the target region is illuminated with extraneous second light, a signal level in the second light image corresponding to the target region.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative configuration of an imaging system configured to capture visible light images and fluorescence images of a scene in which a region of a subject at the scene is illuminated with extraneous light.

FIG. 2 shows a functional diagram of an illustrative extraneous light detection system.

FIG. 3 shows an illustrative method of determining whether a region of a subject is illuminated with extraneous light.

FIG. 4 shows an illustrative visible light image that may be obtained by the extraneous light detection system of FIG. 2 and used to detect extraneous visible light and/or extraneous fluorescence excitation light.

FIGS. 5A and 5B show illustrative graphs that plot adjusted visible light signal levels corresponding to a target region as a function of time.

FIG. 6 shows an illustrative visible light image with a visual notification indicating that a target region is illuminated with extraneous light.

FIG. 7 shows an illustrative visible light image with a visual notification indicating a likely cause of extraneous light.

FIG. 8 shows an illustrative method of performing a mitigation operation for a fluorescence channel of an imaging system when a target region is illuminated with extraneous fluorescence excitation light.

FIG. 9 shows an illustrative method of detecting and mitigating extraneous light.

FIG. 10 shows another illustrative method of detecting and mitigating extraneous light.

FIG. 11 shows an illustrative computer-assisted surgical system.

FIG. 12 shows a functional diagram of an illustrative computing device.

### DETAILED DESCRIPTION

In some situations, a region of a scene may be illuminated with extraneous light. Extraneous light incident on a region of the scene may be received indirectly from a light source of the scene and/or from another source. For example, visible light and/or fluorescence excitation light from the light source may be inter-reflected by an object at the scene, such as a shaft of a surgical instrument at the scene, to a small surface region of tissue at the scene. However, the extraneous light at the region of the scene may adversely affect the visible light images and/or the fluorescence images presented to the user. For example, extraneous fluorescence excitation light incident on a region of tissue increases the intensity of emitted fluorescence as compared with regions of tissue for where there is no extraneous fluorescence excitation light. It is difficult to use the fluorescence channel of the imaging system to detect extraneous fluorescence excitation light because the fluorescence signal varies with time due to the time-varying concentration of fluorophores, photobleaching of the fluorophores, and decay of the emitted fluorescence.

Systems and methods for detecting extraneous light at a scene (e.g., a surgical scene) are described herein. In some examples, an imaging system may capture, over a time period, first light images of a scene illuminated with first light (e.g., light in a first waveband, such as blue light). The first light images depict a subject (e.g., tissue) located at the scene. An extraneous light detection system may track, in the first light images over the time period, pixel values corresponding to a target region of the subject (e.g., a region of surface tissue manually selected by a user or automatically selected by the extraneous light detection system based on image segmentation and/or feature tracking). The extraneous light detection system may determine, based on a comparison of signal levels of pixels that depict the target region with a background model, whether the target region is illuminated with extraneous first light.

As used herein, “first light” is light having a first waveband, which may be a broad, continuous spectrum of light (e.g., white light) or one or more narrowband spectra of light (e.g., one or more color components of light, such as a blue component, a green component, and/or a red component). As also used herein, “first light images” of a scene are generated based on illumination of the scene with first light. For example, first light may be visible light (e.g., blue light or white light) and the first light images (e.g., blue light images or full color images) are generated based on the visible light reflected from the scene.

The background model is representative of reflectivity of the target region for incident first light. For example, the background model may be based on the expected or estimated first light signal levels (pixel values) in the first light images corresponding to the target region when the target region is illuminated with ideal light. In some examples, the target region is illuminated with ideal light when the target region is illuminated with first light from a light source for the scene (e.g., from the distal end of an imaging device) but is not illuminated with extraneous first light. In other examples, the target region is illuminated with ideal light when the target region is illuminated with light modeled by an incident first light model. As explained below, an incident first light model is representative of the spatial distribution of first light emitted from one or more light sources for a scene. The background model may be generated in real time (e.g., over the time period) based on the signal levels of the pixels that depict the target region, may be pre-generated based on first light images captured over one or more prior time periods, and/or may be pre-generated and selected based on a type region (e.g., may be selected for a particular tissue type or anatomical feature).

In some examples, signal levels of portions of the first light images may vary from the background model due to reasons other than incidence of extraneous light (e.g., movement of an imaging device, the light source, and/or the subject).

Accordingly, the extraneous light detection system may adjust (e.g., normalize), based on an incident first light model, the signal levels of pixels in the first light image that depict the target region. Thus, false-positive detections of extraneous light may be avoided.

As used herein, an “incident light model” is representative of a three-dimensional spatial distribution of light from one or more light sources for a scene. An incident light model may be based on or configured for a particular waveband of light, such as a broad, continuous spectrum of light (e.g., white light) or one or more narrowband spectra of light (e.g., one or more color components of light, such as a blue component, a green component, and/or a red component, a NIR component, etc.). For example, an incident first light model (e.g., an incident visible light model) is representative of a three-dimensional spatial distribution of first light (e.g., visible light) from one or more light sources. An incident light model may estimate, or may be used to estimate, an amount of light that is incident on a surface location as a function of position of the surface location with respect to the one or more light sources. For example, an amount of first light (e.g., visible light) that is incident on a surface at a scene may be determined based on a first light image (e.g., visible light image) of the scene and an incident first light model (e.g., incident visible light model) as a function of pixel position and surface depth for each pixel within the first light image (e.g., visible light image).

When the extraneous light detection system determines that extraneous first light is incident on a target region of a subject, the extraneous light detection system may perform one or more mitigation operations. For example, the extraneous light system may provide a notification that the target region is illuminated with extraneous first light and/or may identify and indicate an object that is likely the source of the extraneous first light.

In some examples, the scene may also be illuminated with second light (e.g., light in a second waveband that is different from the first waveband). For example, the second light may be fluorescence excitation light (e.g., NIR light, ultraviolet light (UV), etc.) configured to excite fluorophores present at the scene, which thereby emit fluorescence. The fluorescence may be detected by an imaging system and used to generate fluorescence images.

As used herein, “second light” is light having a second waveband that is different from the first waveband. The second waveband may include a broad, continuous spectrum of light (e.g., white light) or one or more narrowband spectra of light (e.g., one or more color components of light). As also used herein, “second light images” of a scene are generated based on illumination of the scene with second light.

For example, second light may be fluorescence excitation light in a visible or non-visible waveband (e.g., UV or NIR waveband) and second light images (e.g., fluorescence images) are generated based on fluorescence emitted by fluorophores excited by the fluorescence excitation light. The emitted fluorescence may be in a visible or non-visible waveband (e.g., UV or NIR waveband), which may be different from the second waveband.

To illustrate, indocyanine green (ICG) is a fluorophore that, when illuminated with fluorescence excitation light having a wavelength of about 780 nm (second light), emits fluorescence having a wavelength of about 820 nm. An imaging system may detect the emitted fluorescence and generate fluorescence images in which the detected fluorescence signals are false-colored in a visible wavelength (e.g., green). As another example, fluorescein is a fluorophore that, when illuminated with fluorescence excitation light having a wavelength of about 495 nm (second light), emits fluorescence having a wavelength of about 517 nm. Various endogenous fluorophores (e.g., NAD(P)H and FAD) have a peak excitation wavelength in the UV or visible light range and emit fluorescence in the UV and/or visible light range.

In some examples, the signal levels of pixels in the second light images that depict the target region may be adjusted based on an incident second light model to account for non-uniform spatial and/or temporal distribution of second light, which may be caused, for example, by movement of the imaging device, the light source, and/or the subject. An incident second light model (e.g., an incident fluorescence excitation light model) is an incident light model and is representative of a three-dimensional spatial distribution of second light (e.g., fluorescence excitation light) from one or more light sources. An amount of second light (e.g., fluorescence excitation light) that is incident on a surface may be determined based on a second light image (e.g., fluorescence image) and an incident second light model (e.g., fluorescence excitation light model) as a function of pixel position and surface depth for each pixel within the second light image (e.g., fluorescence image).

As with first light, second light from the light source may be inter-reflected by an object at the scene, such as a shaft of a surgical instrument at the scene, to a small surface region of tissue at the scene. The extraneous second light at the region of the scene may adversely affect the second light images. However, in some situations it is difficult to use second light images to determine when the target region is illuminated with extraneous second light. For example, when the second light is fluorescence excitation light and the second images are fluorescence images, the fluorescence signals vary over time due to photobleaching of the fluorophores, decay of the emitted fluorescence, and changing concentration of the fluorophores within the subject.

Accordingly, the extraneous light detection system may infer from the detection of extraneous first light that the target region is also illuminated with extraneous second light (e.g., extraneous fluorescence excitation light). To mitigate the effects of the extraneous second light, the extraneous light detection system may perform a mitigation operation, such as a mitigation operation described above upon detection of extraneous fluorescence excitation light. Additionally or alternatively, the extraneous light detection system may estimate an amount of extraneous second light incident on the target region and may adjust second light signal levels based on the estimated amount of extraneous second light incident on the target region.

Various examples of the systems and methods will be described in detail with reference to the figures. In the examples that follow, first light is described as visible light (e.g., a blue color component of light) and first light images are visible light images generated based on visible light reflected from the scene (e.g., blue light images captured in a blue channel of an imaging system). Additionally, second light is described as fluorescence excitation light (e.g., NIR light or UV light) configured to excite fluorophores at the scene, and second light images are fluorescence images generated based on fluorescence emitted by the excited fluorophores. However, first light and second light may have any other suitable waveband or configuration as may suit a particular implementation. It will be understood that the examples described below are provided as non-limiting examples of how various novel and inventive principles may be applied in various situations. Additionally, it will be understood that other examples not explicitly described herein may also be captured by the scope of the claims set forth below. Systems and methods described herein may provide one or more benefits that will be described or made apparent below.

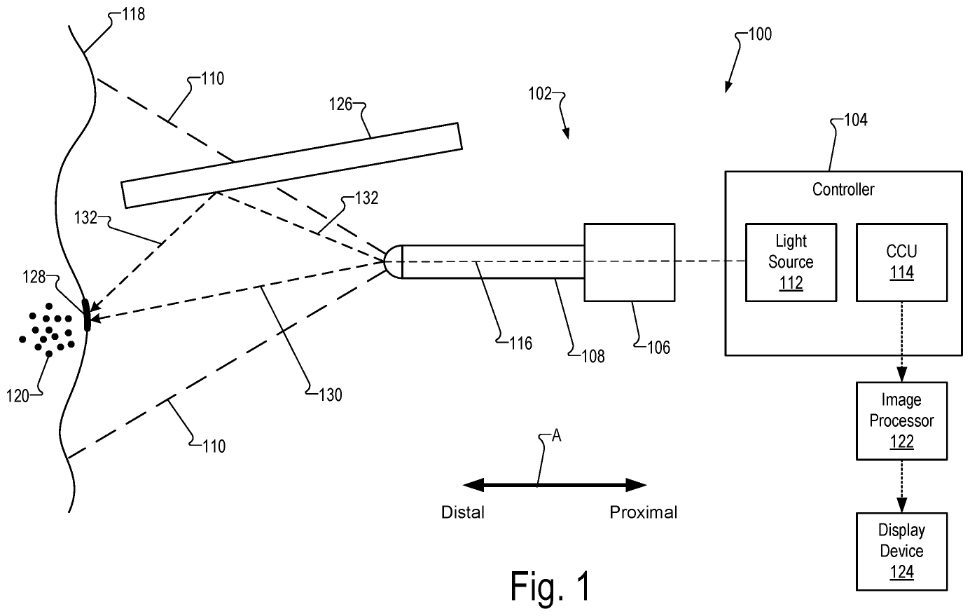

FIG. 1 shows an illustrative configuration of an imaging system 100 configured to capture visible light images (e.g., first light images) and fluorescence images (e.g., second light images) of a scene in which a region of a subject at the scene is illuminated with extraneous light. In some examples, the scene includes an area associated with a subject (e.g., a body) on or within which a fluorescence-guided medical procedure is being performed (e.g., a body of a live human or animal, a human or animal cadaver, a portion of human or animal anatomy, tissue removed from human or animal anatomies, non-tissue work pieces, training models, etc.). In other examples, the scene may be a non-medical scene, such as a scene captured for calibration or operational assessment purposes.

As shown in FIG. 1, imaging system 100 includes an imaging device 102 and a controller 104. Imaging system 100 may include additional or alternative components as may serve a particular implementation, such as various optical and/or electrical signal transmission components (e.g., wires, lenses, optical fibers, choke circuits, waveguides, cables, etc.). While imaging system 100 shown and described herein comprises a visible light imaging system integrated with fluorescence imaging system, imaging system 100 may alternatively be implemented as a standalone visible light imaging system configured to capture only visible light images of the scene.

Accordingly, components of imaging system 100 that function only to capture fluorescence light images may be omitted. In some examples, a visible light imaging system and a fluorescence imaging system may be physically integrated into the same physical components, or a standalone fluorescence imaging system may be inserted into an assistance port of a visible light imaging system.

Imaging device 102 may be implemented by any suitable device configured to capture visible light images and fluorescence images of a scene. Imaging device 102 includes a camera head 106 and a shaft 108 coupled to and extending away from camera head 106. Imaging device 102 may be manually handled and controlled (e.g., by a surgeon performing a surgical procedure on a subject). Alternatively, camera head 106 may be coupled to a manipulator arm of a computer-assisted surgical system and controlled using robotic and/or teleoperation technology. The distal end of shaft 108 may be positioned at or near the scene that is to be imaged by imaging device 102. For example, the distal end of shaft 108 may be inserted into a patient via a cannula. In some examples, imaging device 102 is implemented by an endoscope. As shown by arrow A in FIG. 1, “distal” means situated near or toward the scene or region of interest (e.g., away from controller 104) and “proximal” means situated away from the scene or region of interest (e.g., near or toward controller 104).

Imaging device 102 includes a visible light camera (not shown) configured to capture two-dimensional (2D) or three-dimensional (3D) visible light images of the scene and output visible light image data representative of the visible light images.

Imaging device 102 also include a fluorescence camera (not shown) configured to capture fluorescence images of the scene and output fluorescence image data representative of the fluorescence images. A field of view of imaging device 102 is represented by dashed lines 110. The visible light camera and the fluorescence camera may be implemented by any one or more suitable image sensors configured to detect (e.g., capture, collect, sense, or otherwise acquire) visible light and/or non-visible (e.g., NIR) light, such as a charge coupled device (CCD) image sensor, a complementary metal-oxide semiconductor (CMOS) image sensor, a hyperspectral camera, a multispectral camera, photodetectors based on time-correlated single photon counting (TCSPC) (e.g., a single photon counting detector, a photo multiplier tube (PMT), a single photon avalanche diode (SPAD) detector, etc.), photodetectors based on time-gating (e.g., intensified CCDs), time-of-flight sensors, streak cameras, and the like. In some examples, the visible light camera and/or the fluorescence camera are positioned at the distal end of shaft 108. In alternative examples, the visible light camera and/or the fluorescence camera are positioned closer to the proximal end of shaft 108, inside camera head 106, or outside imaging device 102 (e.g., inside controller 104), and optics included in shaft 108 and/or camera head 106 convey captured light from the scene to the corresponding camera.

Controller 104 may be implemented by any suitable combination of hardware and/or software configured to control and/or interface with imaging device 102. For example, controller 104 may be at least partially implemented by a computing device included in a computer-assisted surgical system. Controller 104 may include a light source 112 and a camera control unit (CCU) 114. Controller 104 may include additional or alternative components as may serve a particular implementation. For example, controller 104 may include circuitry configured to provide power to components included in imaging device 102. In alternative examples, light source 112 and/or CCU 114 are included in imaging device 102 (e.g., in camera head 106). For example, light source 112 may be positioned at the distal end of shaft 108, closer to the proximal end of shaft 108, or inside camera head 106.

Light source 112 is configured to illuminate the scene with light (e.g., visible light and fluorescence excitation light). Light emitted by light source 112 (represented by light ray 116) travels by way of a light channel in shaft 108 (e.g., by way of one or more optical fibers, light guides, lenses, etc.) to a distal end of shaft 108, where the light exits to illuminate the scene. Thus, the light source for the scene is the distal end of imaging device 102. Visible light from light source 112 may include a continuous spectrum of light (e.g., white light) or one or more narrowband color components of light, such as a blue component, a green component, and/or a red component.

Fluorescence excitation light from light source 112 may include one or more broadband spectra of light (e.g., NIR light) or may include one or more narrowband light components (e.g., narrowband NIR light components). Light source 112 may be implemented by any suitable device, such as a flash lamp, laser source, laser diode, light-emitting diode (LED), and the like. While light source 112 is shown to be a single device, light source 112 may alternatively include multiple light sources each configured to generate and emit differently configured light (e.g., visible light and fluorescence excitation light).

Visible light emitted from the distal end of shaft 108 is reflected by a surface 118 of the subject at the scene, and the reflected visible light is detected by the visible light camera of imaging device 102. The visible light camera (and/or other circuitry included in imaging device 102) converts the detected visible light into visible light image data representative of one or more visible light images of the scene. The visible light images may include full color images or may be captured in one or more color channels of imaging system 100 (e.g., a blue color channel).

Fluorescence excitation light emitted from the distal end of shaft 108 excites fluorophores 120 beneath surface 118, which then emit fluorescence that is detected by the fluorescence camera of imaging device 102. The fluorescence camera (and/or other circuitry included in imaging device 102) converts the detected fluorescence into fluorescence image data representative of one or more fluorescence images of the scene. Imaging device 102 transmits the visible light image data and the fluorescence image data via a wired or wireless communication link to CCU 114.

CCU 114 may be configured to control (e.g., define, adjust, configure, set, etc.) operation of the visible light camera and fluorescence camera and is configured to receive and process the visible light image data and the fluorescence image data. For example, CCU 114 may packetize and/or format the visible light image data and the fluorescence image data. CCU 114 outputs the visible light image data and the fluorescence image data to an image processor 122 for further processing. While CCU 114 is shown to be a single unit, CCU 114 may alternatively be implemented by multiple CCUs each configured to control distinct image streams (e.g., a visible light image stream and a fluorescence image stream).

Image processor 122 may be implemented by one or more computing devices external to imaging system 100, such as one or more computing devices included in a computer-assisted surgical system. Alternatively, image processor 122 may be included in imaging system 100 (e.g., in controller 104). Image processor 122 may prepare visible light image data and/or fluorescence image data for display (e.g., in the form of one or more still images and/or video streams) by a display device 124 (e.g., a computer monitor, a projector, a tablet computer, or a television screen). For example, image processor 122 may false-color fluorescing regions (e.g., green, yellow, blue, etc.) and/or selectively apply a gain to adjust (e.g., increase or decrease) the intensity of the fluorescing regions. Image processor 122 may also generate a graphical overlay based on fluorescence image data and combine the graphical overlay with a visible light image to form an augmented image (e.g., a visible light image augmented with fluorescence image data). Image processor 122 may also adjusted the visible light image to correct various image parameters, such as auto-exposure, gain, and/or white balance.

In some examples, image processor 122 may adjust the fluorescence image data to account for non-uniform spatial and/or temporal distribution of fluorescence excitation light. Such non-uniform distributions of fluorescence excitation light may occur, for example, when imaging device 102 changes position and/or orientation with respect to surface 118. Additionally, the intensity of the fluorescence excitation light may fall off with distance and/or angle from the light source (e.g., a distal end of imaging device 102). The non-uniform distributions of fluorescence excitation light result in corresponding variations in the detected fluorescence signal since the intensity of emitted fluorescence is proportional to the intensity of incident fluorescence excitation light.

To account for such variations, image processor 122 may adjust (e.g., normalize) the detected fluorescence signal for a portion of a fluorescence image with respect to a measure of fluorescence excitation light estimated to be incident on surface 118 corresponding to such image portion. Such estimated measure of fluorescence excitation light may be determined, for example, using one or more sensors and/or an incident fluorescence excitation light model. Thus, the adjusted output fluorescence signal is substantially independent of any fluorescence signal variations attributed to the non-uniform distribution of fluorescence excitation light.

Illustrative systems and methods for adjusting fluorescence signal levels for a portion of an image with respect to a measure of fluorescence excitation light estimated to be incident on a surface corresponding such portion are described in U.S. Patent Application Publication No. 2022/0015857, published on Jan. 20, 2022, which is incorporated herein by reference in its entirety.

As mentioned, a region of surface 118 may be illuminated with extraneous light. As shown in FIG. 1, a region 128 of surface 118 is illuminated with visible light and/or NIR light directly from imaging device 102, as shown by light ray 130. However, an object 126 (e.g., a surgical instrument) located at the scene also reflects some visible light and/or NIR light, as shown by light ray 132, from imaging device 102 toward region 128. Thus, region 128 is illuminated with extraneous visible light and/or extraneous NIR fluorescence excitation light. As used herein, “extraneous light” refers to light (e.g., visible light and/or NIR light) that is incident on a region of tissue and that is received indirectly from a light source of the scene (e.g., by inter-reflections within the scene) or from another source (e.g., external light leakage). As will be explained in more detail below, “extraneous” light may also refer to light not modeled by an incident light model that may be used when processing visible light images to detect extraneous light.

Extraneous visible light may adversely affect visible light images. For example, region 128 may appear whitewashed or saturated. In some examples, extraneous visible light at region 128 may affect video pipeline processing, such as autoexposure processing and/or white balance processing. Extraneous visible light at region 128 may also cause an undesirable amount of optical energy to be concentrated on surface 118 at region 128.

Extraneous fluorescence excitation light may also adversely affect fluorescence images. For example, a greater amount of fluorescence excitation light incident on region 128 of surface 118 may result in a greater amount of fluorescence emitted from fluorophores underneath surface 118 at region 128. Accordingly, the detected fluorescence signal from region 128 may not accurately represent the concentration of fluorophores beneath region 128 and, hence, may not accurately represent or indicate a state of the medium (e.g., tissue) in which the fluorophores are located.

Extraneous fluorescence excitation light may also adversely affect the processing of detected fluorescence signals to compensate for non-uniform spatial and/or temporal distribution of fluorescence excitation light. For example, extraneous fluorescence excitation light at region 128 may adversely affect the measure of fluorescence excitation light estimated to be incident on surface 118 and, thus, may result in inaccurate adjustment of the fluorescence signal. Thus, the adjusted fluorescence signal levels that are displayed on display device 124 may not accurately represent or indicate a state of the tissue.

An extraneous light detection system is configured to determine whether a target region of a subject is illuminated with extraneous light. If the target region is illuminated with extraneous light, the extraneous light detection system may perform a mitigation operation, such as providing a notification and/or adjusting a signal affected by the extraneous light.

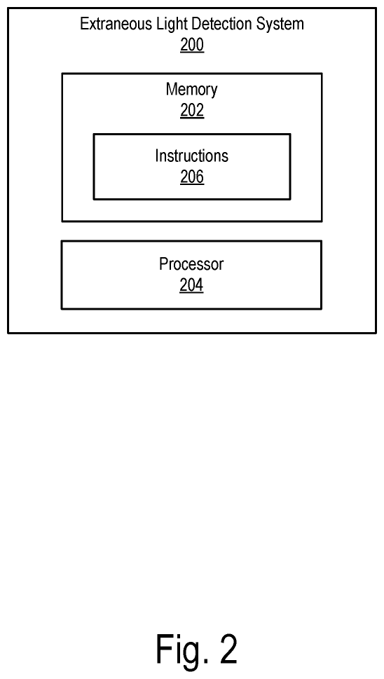

FIG. 2 shows a functional diagram an illustrative extraneous light detection system 200 (“system 200”). System 200 may be included in, implemented by, or connected to an imaging system, a surgical system, an image processor, and/or a computing system described herein. For example, system 200 may be implemented, in whole or in part, by imaging system 100, image processor 122, a computer-assisted surgical system, and/or a computing system communicatively coupled to an imaging system or a computer-assisted surgical system.

As shown, system 200 includes, without limitation, a memory 202 and a processor 204 selectively and communicatively coupled to one another. Memory 202 and processor 204 may each include or be implemented by hardware and/or software components (e.g., processors, memories, communication interfaces, instructions stored in memory for execution by the processors, etc.). For example, memory 202 and processor 204 may be implemented by any component in a computer-assisted surgical system. In some examples, memory 202 and processor 204 may be distributed between multiple devices and/or multiple locations as may serve a particular implementation.

Memory 202 may maintain (e.g., store) executable data used by processor 204 to perform any of the operations described herein. For example, memory 202 may store instructions 206 that may be executed by processor 204 to perform any of the operations described herein. Instructions 206 may be implemented by any suitable application, software, code, and/or other executable data instance. Memory 202 may also maintain any data received, generated, managed, used, and/or transmitted by processor 204.

Processor 204 may be configured to perform (e.g., execute instructions 206 stored in memory 202 to perform) various operations associated with determining whether a region of a subject is illuminated with extraneous light. For example, processor 204 may access visible light images of a scene illuminated with visible light and captured over a time period, the visible light images depicting a subject located at the scene. Processor 204 may track, in the visible light images over the time period, pixel values of a target region of the subject. Processor 204 may determine, based on a comparison of signal levels of pixels that depict the target region with a background model representative of reflectivity of the target region, whether the target region is illuminated with extraneous visible light. Illustrative operations that may be performed by processor 204 will be described herein. In the description that follows, any references to operations performed by system 200 may be understood to be performed by processor 204 of system 200.

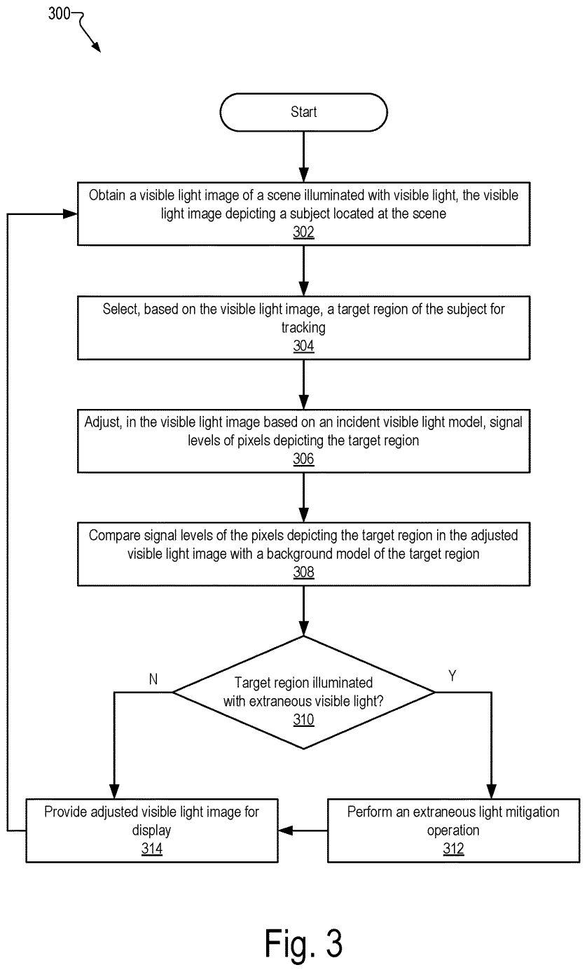

FIG. 3 shows an illustrative method 300 of determining whether a target region of a subject is illuminated with extraneous visible light. While FIG. 3 shows operations according to one embodiment, other embodiments may omit, add to, reorder, and/or modify any of the operations shown in FIG. 3. One or more of the operations shown in FIG. 3 may be performed by system 200, by any components included therein, and/or by any implementation thereof. Operations of method 300 may be performed in any suitable way, including any way described herein.

At operation 302, system 200 obtains a visible light image of a scene. The visible light image depicts a subject located at the scene. In some examples, the visible light image is captured in a narrowband color channel of the imaging system (e.g., a blue channel). As explained below, signal levels of pixels in the visible light image that depict a target region of the subject are compared with a background model representative of reflectivity of the target region to determine if the target region is illuminated with extraneous visible light. In the examples that follow, the target region is a selected region of the subject that is depicted by a subset of multiple pixels of the visible light image (e.g., a portion of the visible light image). However, in other examples that will be described later, the target region may be a region of the subject that is depicted by a single pixel of the visible light image, and system 200 may process all pixels of the visible light image (or a portion of the visible light image) using, e.g., dense optical flow to track all target regions.

At operation 304, system 200 selects, based on the visible light image, a target region of the subject to monitor for extraneous light. If a target region was previously selected, system 200 continues with the previously selected target region. If a target region was not previously selected, or if an additional target region is to be selected, system 200 selects a new target region. Once a target region of the subject has been selected, system 200 may use image feature tracking to track pixel values of the target region in subsequently captured visible light images of the scene. System 200 may select a new target region in any suitable way, including based on active or passive manual input provided by a user or automatically (e.g., without any active or passive manual input).

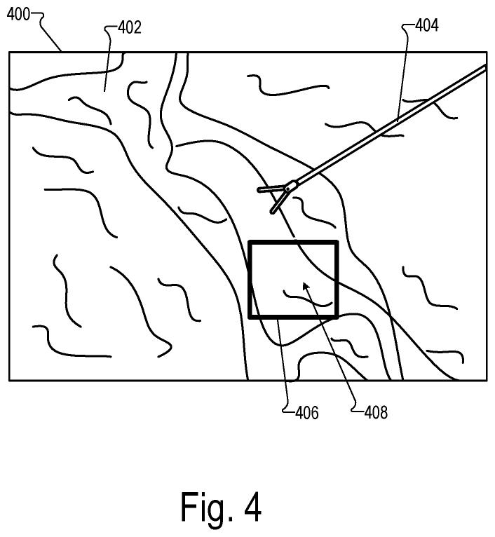

In some examples, system 200 selects a target region based on manual input provided by a user by way of the visible light image. FIG. 4 shows an illustrative visible light image 400 that may be obtained by system 200 and that may be used to select a target region. As shown, visible light image 400 depicts a scene including tissue 402 and a surgical instrument 404. A user may draw a box 406 (or circle, oval, freeform drawing, or other shape) on visible light image 400 to select the region 408 of tissue depicted in box 406 as the target region. Box 406 may be drawn in any suitable way, such as with a real or virtual instrument, a cursor, or a movable box 406.

In other examples, system 200 may select a target region based on image segmentation. For example, a user may indicate (e.g., a form of active manual input) an object located at the scene (e.g., an anatomical feature), and system 200 may use image segmentation to select the indicated object as the target region. The user may indicate the object in any suitable way, such as by drawing a box (e.g., box 406 or other shape) around the object or selecting the object with a real or virtual instrument or a cursor.

In further examples, system 200 may determine, based on a user's eye gaze (e.g., a form of passive manual input), a portion of the visible light image that the user is presently viewing and may select a region of the subject corresponding to the viewed portion of visible light image. To illustrate with respect to FIG. 4, system 200 may select, as target region 408, an object (e.g., anatomical feature) that the user is presently viewing. Alternatively, system 200 may select, as target region 408, the region of tissue 402 (e.g., a rectangular or circular region) within the center of the field of vision of the user.

In some examples, system 200 may automatically select the object without any user input (active or manual) provided by way of the visible light image and/or during the current surgical procedure. For instance, system 200 may determine, based on surgical procedure data provided previously to performing the current surgical procedure, a type of surgical procedure being performed and may automatically identify, by image segmentation and image recognition, a particular object associated with the particular type of surgical procedure.

Additionally or alternatively, system 200 may automatically select the object based on a pre-operative image (e.g., a prior endoscopic image, MRI scan, or CT scan) that is registered to the surgical scene. For example, a pre-operative image may indicate the location of a tumor at the surgical scene. The pre-operative image may be registered to the surgical scene in any suitable way. Based on the pre-operative image, system 200 may automatically select a region of the subject corresponding to the location of the tumor indicated in the pre-operative image.

Referring again to FIG. 3, at operation 306, system 200 adjusts, in the visible light image based on an incident visible light model, signal levels of pixels depicting the target region. The incident visible light model may be used, along with pixel position and depth data, to adjust (e.g., normalize) the visible light signal levels to account for variations in the detected visible light signal levels attributed to variations in incident light on the surface. Variations in incident visible light on the surface are generally caused by something other than extraneous light, such as uneven distribution patterns, movement of tissue, and/or movement of the light source.

As explained above, the incident visible light model is representative of the three-dimensional spatial distribution of visible light emitted from a light source (e.g., the distal end of imaging device 102). For example, a theoretical and/or empirical model (e.g., a depth map) may provide information on how the intensity of visible light from the light source falls off with distance and/or angle from the light source. Such an incident visible light model may be used to determine the amount of visible light incident at a particular surface location within the field of view of the imaging device. The incident visible light model can be configured to, for example, account for the intensity variation due to the particular illumination pattern of the light source. In some examples, the imaging system (e.g., imaging system 100) may include a distance sensor (for example, a distance sensor disposed at the distal end of imaging device 102) for measuring and/or estimating a distance of the light source from the surface tissue in the field of view. The amount of visible light incident on the various surface regions in the field of view of the imaging device may be determined or estimated using the incident visible light model and pixel position and distance information.

System 200 may adjust signal levels of pixels in the visible light image that depict the target region based on the amount of visible light that is estimated to be incident on the target region. In some implementations, such correction can be represented using the following equation (1):

$$[ {VLS}_{adjusted} ] = [ {VLS}_{detected} ] / [ {IVL}_{estimated} ] \qquad ( 1 )$$

where VLS_{detected} represents the visible light signal detected (e.g., captured by the visible light camera for one or more pixels corresponding to the target region, IVL_{estimated} represents the estimated amount of visible light that is incident on the target region, and VLS_{adjusted} represents the adjusted visible light signal level for the one or more pixels corresponding to the target region. Other adjustment and/or normalization schemes may be used as may suit a particular implementation. Although not shown in FIG. 3, the detected visible light signal may also be adjusted to correct other image parameters, such as auto-exposure, gain, and/or white balance.

The adjusted visible light image accounts for variations in reflected visible light signal levels due to the variations in the amount of incident visible light (attributable to, for example, different distances and/or orientations with respect to the light source) and provides a more accurate representation of the reflected visible light (and tissue reflectivity). Additionally, the adjusted visible light image isolates variations in reflected visible light signal levels due to extraneous light, thereby preventing false-positive detection of extraneous light.

At operation 308, system 200 compares signal levels of pixels depicting the target region in the adjusted visible light image with a background model that is representative of reflectivity of the target region.

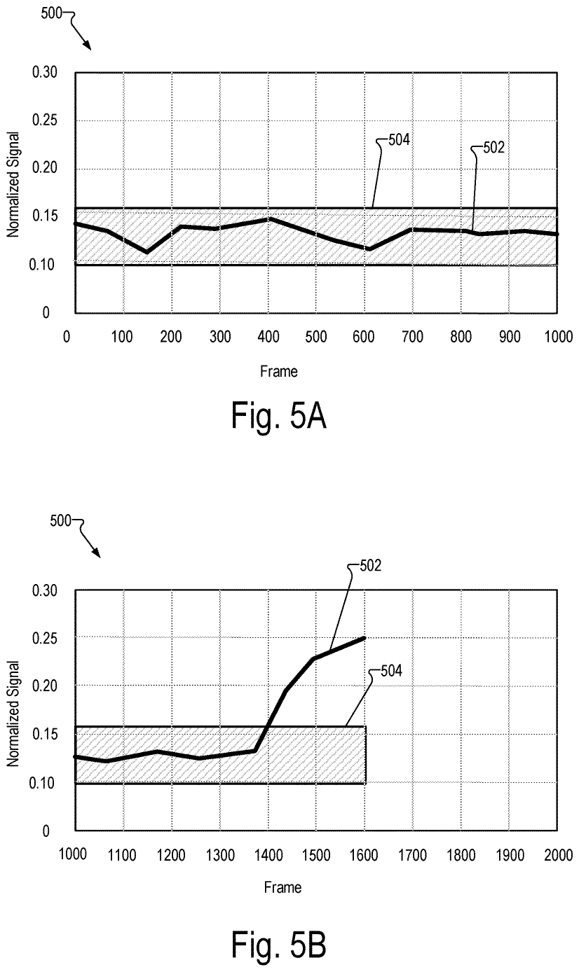

The background model may be generated in any suitable way. In some examples, the background model is generated in real-time based on visible light images captured over a period of time. In some examples, the period of time includes the current time (e.g., the visible light images includes the current or most recent visible light image). FIGS. 5A and 5B show an illustrative graph 500 that plots the adjusted visible light signal levels corresponding to a target region (e.g., target region 408) as a function of time (e.g., for each visible light image in a visible light image stream). A curve 502 represents the adjusted visible light signal levels corresponding to the target region over a period of time ranging from frame 1 to frame 1000. Curve 502 may be generated based on an average, median, minimum, sum, or any other statistical analysis of the signal levels of all pixels corresponding to target region 408. System 200 may generate a background model 504 based on curve 502. Background model 504 indicates a range of an expected or estimated normalized signal level corresponding to the target region. In the example of FIG. 5A, the signal levels of background model 504 range from approximately 0.10 to approximately 0.16. System 200 may generate background model 504 in any suitable way using any suitable statistical analysis, such as a signal valley detector or an ordered statistic, to ensure that background model 504 is not influenced by extraneous light. Thus, in some examples background model 504 is implicitly a model of reflectivity of tissue 402.

In some examples, the background model may additionally or alternatively be generated based on one or more visible light images captured prior to the current period of time (e.g., during one or more procedures performed prior to the current surgical procedure). For example, background model 504 may be generated based on visible light images captured during a pre-operative procedure performed on the same subject. In some examples, the pre-operatively generated background model may be used as a baseline model and may be updated in real-time based on visible light images captured during the current period of time (e.g., during the current surgical procedure).

In further examples, the background model may be generated based on visible light images captured during multiple different procedures performed on multiple different subjects. Such background model may be used as a baseline model and may be updated in real-time based on visible light images captured during the current period of time (e.g., during the current surgical procedure).

In yet further examples, the background model may be specific to the particular type of tissue of the target region. For example, system 200 may determine the type of tissue of the target region and select, from multiple different background models each associated with a distinct type of tissue, a background model associated with the type of tissue of the target region. System 200 may determine the type of tissue in any suitable way. For example, system 200 may use image recognition or computer vision to determine a type of anatomical feature and thereby determine a tissue type based on the anatomical feature. As another example, system 200 may determine the type of tissue based on surgical procedure data, such as data indicating a type of procedure being performed (e.g., a hysterectomy, a hernia repair, a biopsy, a tumor resection, etc.). In such examples, system 200 may assume that tissue at the surgical scene is a particular type of tissue associated with the particular surgical procedure. In yet further examples, system 200 may determine the type of tissue based on a pre-operative image registered to the surgical scene, as explained above. A background model for each particular type of tissue may be generated in any suitable way. In some examples, the background model may be generated empirically from one or more distinct procedures performed on one or more subjects.

Returning to FIG. 3, at operation 308 system 200 compares signal levels of pixels depicting the target region in the adjusted visible light image (as adjusted at operation 306) with the background model to determine whether the target region is illuminated with extraneous light. System 200 may determine that the target region is illuminated with extraneous light in any suitable way. For example, system 200 may determine that the target region is illuminated with extraneous light when the adjusted signal levels (e.g., the average, median, maximum, minimum, or sum of the signal levels) of the pixels corresponding to the target region exceed or fall outside the background model for a threshold period of time (e.g., 3 seconds, 5 seconds, 50 frames, 100 frames, etc.), and/or exceed the background model by a threshold amount (e.g., by 10%, by 25%, etc.).

To illustrate, FIG. 5B shows graph 500 over a time period ranging from frame 1000 to a current frame (frame 1600). As indicated by curve 502, the adjusted visible light signal level corresponding to the target region exceeds background model 504 beginning at frame 1400 until at least the current time (frame 1600). System 200 may determine that the target region is illuminated with extraneous light when the adjusted signal levels corresponding to the target region exceed background model 504 (e.g., beginning at time 1400), exceed background model 504 for a threshold period of time (e.g., 100 frames, beginning at frame 1500), and/or exceed background model 504 by a threshold amount (e.g., by 25%, which begins at about frame 1450).

Referring again to FIG. 3, at operation 310, if system 200 determines that the target region is illuminated with extraneous light, system 200 proceeds to operation 312. At operation 312, system 200 performs an extraneous light mitigation operation to mitigate the effects of the extraneous light at the target region. Illustrative mitigation operations will be described below in more detail.

If system 200 determines that the target region is not illuminated with extraneous light, or after system 200 performs an extraneous light mitigation operation at operation 312, processing of method 300 proceeds to operation 314.

At operation 314, system 200 provides the adjusted visible light image for display by a display device (e.g., display device 124). In some examples, as will be described below in more detail, the visible light image may be combined with a fluorescence image to produce an augmented image (e.g., a visible light image overlaid with fluorescence signals), and the augmented image may be provided for display by the display device. Processing of method 300 then returns to operation 302 to obtain a subsequently-captured visible light image and repeats method 300 for the target region.

Thus, system 200 may obtain visible light images of a scene illuminated with visible light and captured over a time period, the visible light images depicting a subject located at the scene; track, in the visible light images over the time period, a target region of the subject; and determine, based on a comparison of signal levels of pixels that depict the target region with a background model representative of reflectivity of the target region, whether the target region is illuminated with extraneous visible light.

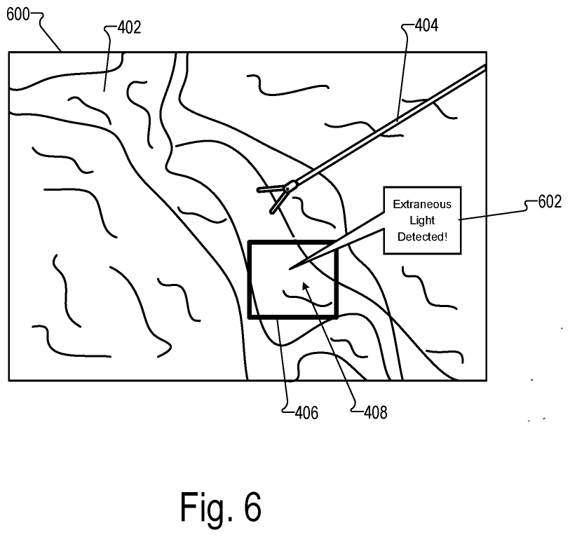

Illustrative extraneous light mitigation operations will now be described. In some examples, an extraneous light mitigation operation includes providing a notification that the target region is illuminated with extraneous light. The notification may have any form, such as a visual, audible, and/or a haptic notification (which may be provided by way of a user control system). The visual notification may include, for example, a message, a warning icon, and/or a graphical element overlaid on the visible light image and/or within a peripheral region of a graphical user interface (GUI) in which the visible light image is displayed. FIG. 6 shows an illustrative visible light image 600 with a visual notification. Visible light image 600 is similar to visible light image 400 except that, in visible light image 600, a visual notification 602 is overlaid on visible light image 600 to indicate to the user that target region 408 is illuminated with extraneous visible light. While FIG. 6 shows that visible light image 600 displays box 406, in other examples box 406 may be omitted or may be hidden and may be toggled on and off as desired by a user. System 200 may continue to overlay visual notification 602 on visible light image 600 until system 200 determines that target region 408 is no longer illuminated with extraneous visible light.

In further examples, an extraneous light mitigation operation includes identifying an object at the surgical scene that is a likely cause of the extraneous light at the target region and indicating the object. System 200 may identify the object in any suitable way. In some examples, system 200 identifies the object based on kinematic data representative of operations of one or more objects located at the scene during the time period, such as one or more robotic-assisted surgical instruments. To illustrate with reference to the example of FIGS. 4 and 5B, system 200 may determine, based on kinematic data and/or image tracking, that surgical instrument 404 changed a pose (e.g., a position and/or orientation within the scene) immediately prior to detection of extraneous light (e.g., at frame 1400). Thus, system 200 may determine that surgical instrument 404 is likely the cause of the extraneous light at target region 408.

Accordingly, system 200 may indicate surgical instrument 404 as the likely cause of the extraneous light.

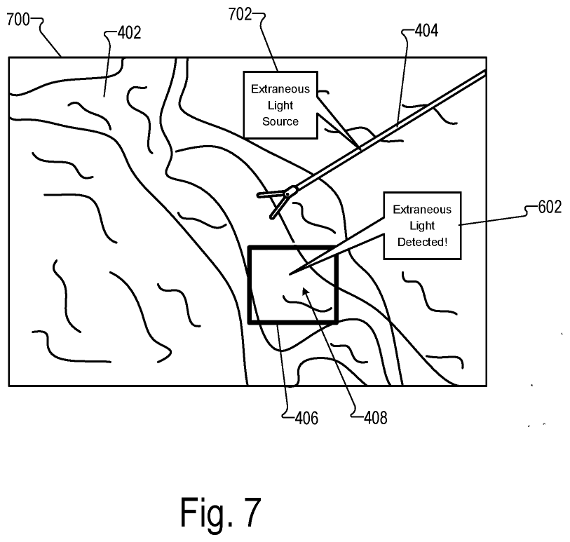

The indication of the object may be provided in addition to or alternatively to the visual notification described above and may have any suitable form. For example, the indication may include a graphical element (e.g., an arrow), a message, and/or false-coloring of the object. FIG. 7 shows an illustrative visible light image 700 in which a likely cause of the extraneous light is indicated. Visible light image 700 is similar to visible light image 600 except that, in visible light image 700, a visual indication 702 is overlaid on visible light image 700 to indicate that surgical instrument 404 is the likely cause of the extraneous light.

In some examples, system 200 may be unable to identify an object that is likely the cause of the extraneous light. Accordingly, system 200 may abstain from indicating any object as a likely cause of the extraneous light. Alternatively, system 200 may provide a notification that the cause of the extraneous light cannot be determined.

Various modifications may be made to method 300 described above. In some examples, operation 306 may be omitted if system 200 determines that the light source (e.g., imaging device 102) and any objects at the scene (e.g., object 126) have not moved and/or that visible light intensity output by the light source has not changed. For example, system 200 may determine, based on image segmentation (e.g., feature tracking) and/or kinematic data, that imaging device 102 and object 126 have not changed their pose relative to an immediately prior frame, or that the amount of any change in pose is less than a threshold amount. Accordingly, system 200 may infer that there are no variations in the distribution of light due to movement of imaging device 102 and object 126 and may omit adjusting the visible light image based on an incident visible light model.

In some examples, system 200 may perform method 300 for each of multiple different target regions. For example, system 200 may track, over a time period, multiple different target regions of the subject to determine whether any one of the target regions is illuminated with extraneous visible light. In some examples, system 200 generates or selects a distinct background model for each target region. If system 200 determines that any one of the target regions is illuminated with extraneous visible light, system 200 may perform a mitigation operation for that target region. Alternatively, system 200 may perform a mitigation operation when a threshold number of target regions are determined to be illuminated with extraneous visible light.

In the examples of method 300 described above, the target region of the subject is depicted by a subset of pixels of the visible light image, and system 200 processes the target region as a whole (e.g., based on an average pixel value or some other combined statistical value of all pixels corresponding to the target region). In alternative examples, the target region is a region of the subject that is depicted by a single pixel of the visible light image, and system 200 processes multiple target regions on an individual pixel basis for every pixel within the visible light image or within a selected portion of the visible light image (e.g., within box 406 or other portion of the visible light image selected as described above). For example, system 200 may perform method 300 for each pixel of the entire visible light image or selected portion of the visible light image using dense optical flow to track each target region, and system 200 may determine, for each pixel, if the corresponding target region is illuminated with extraneous visible light. In examples in which system 200 processes all pixels of the visible light image, operation 304 may be omitted since each pixel will be analyzed in method 300.

When system 300 processes multiple target regions on an individual pixel basis, as just described, system 200 may perform an extraneous light mitigation operation when any target region is determined to be illuminated with extraneous visible light, or when a threshold number of pixels have signal levels that exceed or fall outside of the corresponding background model, exceed or fall outside the corresponding background model for a threshold period of time (e.g., 3 seconds, 5 seconds, 50 frames, 100 frames, etc.), and/or exceed or fall outside the corresponding background model by a threshold amount (e.g., by 10%, by 25%, etc.).

In some examples, system 200 may use the principles described above to measure reflectivity of tissue. For example, system 200 may perform operations 302 to 306 of method 300 and use the adjusted visible light images to build a model of reflectivity of tissue. As mentioned, the visible light signal levels of the visible light images are adjusted (e.g., normalized) based on an incident visible light model to thereby account for variations in the visible light signal levels caused by the three-dimensional spatial variations in the distribution of visible light at the scene.

In the examples described above, system 200 may perform method 300 to determine whether the target region is illuminated with extraneous visible light. When system 200 determines that the target region is illuminated with extraneous visible light, system 200 may infer that the target region is also illuminated with extraneous fluorescence excitation light. Thus, the visible light color channel of the imaging system may be used to detect extraneous fluorescence excitation light at a target region of the scene. Using the visible light color channel of the imaging system to detect extraneous fluorescence excitation light has the advantage that the detection of extraneous fluorescence excitation light is based on the reflectivity of the tissue, which is generally stable over time. In contrast, it is difficult to use the fluorescence channel of the imaging system to detect extraneous fluorescence excitation light since emitted fluorescence is not fixed but changes with time due to photobleaching of the fluorophores, decay of emitted fluorescence, and changing concentration of the fluorophores in the subject.

When system 200 determines that the target region is illuminated with extraneous fluorescence excitation light, system 200 may perform any mitigation operation described above with reference to extraneous visible light. Additionally, system 200 may perform a mitigation operation configured to mitigate the effects of extraneous fluorescence excitation light. For example, as will now be described, a mitigation operation may include estimating an amount of extraneous fluorescence excitation light incident on the target region and adjusting a fluorescence image based on the estimated amount of extraneous fluorescence excitation light incident on the target region.

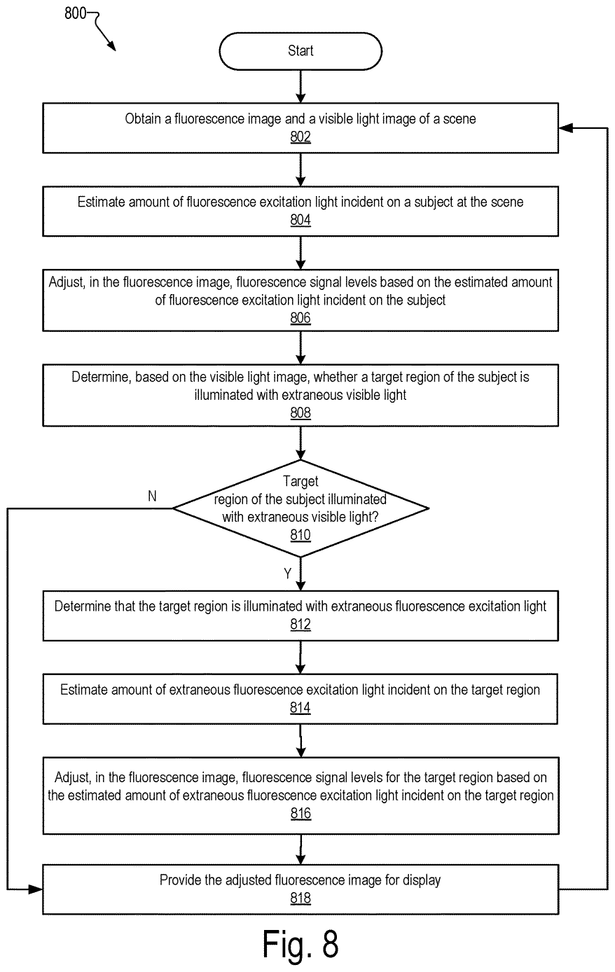

FIG. 8 shows an illustrative method 800 of performing a mitigation operation for a fluorescence channel of an imaging system when a target region is illuminated with extraneous fluorescence excitation light. While FIG. 8 shows operations according to one embodiment, other embodiments may omit, add to, reorder, and/or modify any of the operations shown in FIG. 8. One or more of the operations shown in FIG. 8 may be performed by system 200, by any components included therein, and/or by any implementation thereof. Operations of method 800 may be performed in any suitable way, including any way described herein.

At operation 802, system 200 obtains a fluorescence image and a visible light image of a scene. The fluorescence image is captured based on fluorescence emitted from a subject located at the scene and the visible light image is captured based on visible light reflected from the subject. In some examples, the fluorescence image and the visible light image were captured at substantially the same time so that they represent the same state of the scene.

At operation 804, system 200 estimates the amount of fluorescence excitation light that is incident on the subject corresponding to each pixel of the fluorescence image. As explained above, the non-uniform spatial and/or temporal distribution of fluorescence excitation light may result in corresponding variations in the detected fluorescence signal. System 200 may estimate the amount of fluorescence excitation light that is incident on the subject in any suitable way. For example, the amount of fluorescence excitation light that is incident on the subject is sensed using one or more sensors and/or based on an incident fluorescence excitation light model, as described above.

At operation 806, system 200 adjusts, in the fluorescence image, the fluorescence signal levels of the fluorescence image based on the estimated amount of fluorescence excitation light that is incident on the subject. By adjusting the fluorescence signal levels in this manner, the fluorescence signal for various portions of a fluorescence image may be normalized with respect to the amount of fluorescence excitation light estimated to be incident on the subject corresponding to such portions of the fluorescence image. The normalized fluorescence images account for variations in fluorescence signal due to the variations in the amount of incident fluorescence excitation energy (attributable to, for example, different distances and/or orientations with respect to the fluorescence excitation light source, etc.), and may provide a more accurate representation of the underlying fluorescence. Thus, the adjusted fluorescence signal levels are substantially independent of the variations due to the non-uniform distribution of the fluorescence excitation light over time.

In some implementations, the adjustment may be represented using the following equation (2):

$$[ {FS}_{adjusted} ] = [ {FS}_{detected} ] / [ {IFEL}_{estimated} ] \qquad ( 2 )$$

where FS_{detected} represents the fluorescence signal detected (e.g., captured) by the fluorescence camera for one or more pixels corresponding to the target region, IFEL_{estimated} represents the estimated amount of fluorescence excitation light that is incident on the target region, and FS_{adjusted} represents the adjusted fluorescence signal level for the one or more pixels corresponding to the target region. Other adjustment and/or normalization schemes may be used as may suit a particular implementation.

Although not shown in FIG. 8, the fluorescence signal levels may also be adjusted to correct other image parameters, such as gain.

At operation 808, system 200 determines, based on the visible light image obtained in operation 802, whether a target region of the subject is illuminated with extraneous fluorescence excitation light. Operation 808 may be performed in any suitable way, such as by performing method 300 (e.g., operations 302 to 310).

At operation 810, if system 200 determines that the target region is illuminated with extraneous visible light, processing of method 800 proceeds to operation 812. If system 200 determines that the target region is not illuminated with extraneous visible light, processing of method 800 skips operations 812 to 816 and proceeds to operation 818.

At operation 812, system 200 determines, based on the determination that the target region is illuminated with extraneous visible light, that the target region is also illuminated with extraneous fluorescence excitation light and proceeds, in operations 814 and 816, to perform an extraneous light mitigation operation. System 200 may determine that the target region is also illuminated with extraneous fluorescence excitation light in any suitable way. In some examples, system 200 determines that the target region is illuminated with extraneous fluorescence excitation light in response to a determination that the target region is illuminated with extraneous visible light. In other examples, system 200 determines that the target region is illuminated with extraneous fluorescence excitation light in response to a determination that the target region is illuminated with extraneous visible light and further in response to a determination that the extraneous visible light exceeds a threshold amount and/or persists for a threshold duration of time. In further examples, system 200 determines that the target region is illuminated with extraneous fluorescence excitation light based on identification of a source of the extraneous visible light. For example, if the source of the extraneous visible light (e.g., an instrument shaft) is known by system 200 to not reflect fluorescence excitation light (e.g., absorbs substantially all NIR light), or is a secondary light source of only visible light, system 200 does not determine that the target region is illuminated with extraneous fluorescence excitation light.

At operation 814, system 200 estimates an amount of extraneous fluorescence excitation light incident on the target region. In some examples, system 200 estimates the amount of extraneous fluorescence excitation light incident on the target region by estimating the amount of extraneous visible light incident on the target region. In some examples, system 200 determines the amount of extraneous visible light incident on the target region based on the visible light image (e.g., based on a comparison of the signal levels of the pixels depicting the target region in the adjusted visible light image with the background model, as in operation 308 of method 300). For instance, in the example of FIG. 5B, system 200 may determine that, at the current time (frame 1600), the intensity of extraneous visible light (having a normalized signal level of 0.25) is approximately 56% greater than the upper threshold level of background model 504 (a signal level of 0.16).

In alternative examples, system 200 determines the amount of extraneous visible light based on a comparison of the current visible light image with one or more previously-captured visible light images depicting the target region. For instance, using again the example of FIG. 5B, system 200 may determine that, at the current time (frame 1600), the intensity of extraneous visible light (having a normalized signal level of 0.25) is approximately 100% greater than a running average normalized signal level (approximately 0.125) for the target region over frames 1 to 1400. System 200 may then determine the amount of extraneous fluorescence excitation light incident on the target region based on the amount of extraneous visible light incident on the target region.

At operation 816, system 200 adjusts the fluorescence signal levels of the fluorescence image corresponding to the target region based on the estimated amount of extraneous fluorescence excitation light incident on the target region. The fluorescence signal adjustment may be based on a correlation between the amount of extraneous fluorescence excitation light and the resulting increased fluorescence signal. The correlation may be, for example, a one-to-one ratio or some other correlation that may be determined theoretically or empirically. For example, if system 200 determines that the intensity of extraneous visible light is approximately 56% greater than the upper threshold level of background model 504, system 200 may reduce the fluorescence signal levels for the target region by 56%. In some examples, system 200 may adjust the fluorescence signal levels for the target region to a level indicated by the background model.

At operation 818, system 200 provides the adjusted fluorescence image for display by a display device. In some examples, the adjusted fluorescence image is combined with the visible light image (provided at operation 314 of method 300) to present an augmented image (e.g., the visible light image overlaid with the adjusted fluorescence signals). Processing of method 800 then returns to operation 302 to access a subsequently-captured fluorescence image and visible light image and repeats method 800 for the target region.

In the examples of method 800 described above, the target region of the subject is depicted by a subset of pixels of the visible light image, and system 200 processes the target region as a whole (e.g., based on an average pixel value or some other combined statistical value of all pixels corresponding to the target region). In alternative examples, the target region is a region of the subject that is depicted by a single pixel of the visible light image, and system 200 processes multiple target regions on an individual pixel basis for every pixel within the visible light image or within a selected portion of the visible light image (e.g., within box 406 or other portion of the visible light image selected as described above). For example, system 200 may perform method 800 for each pixel of the entire fluorescence image or selected portion of the fluorescence image to determine, for each pixel, if the corresponding target region is illuminated with extraneous fluorescence light. For each target region that is determined to be illuminated with extraneous fluorescence excitation light, system 200 may adjust the corresponding fluorescence signal level as described in operations 814 and 816.

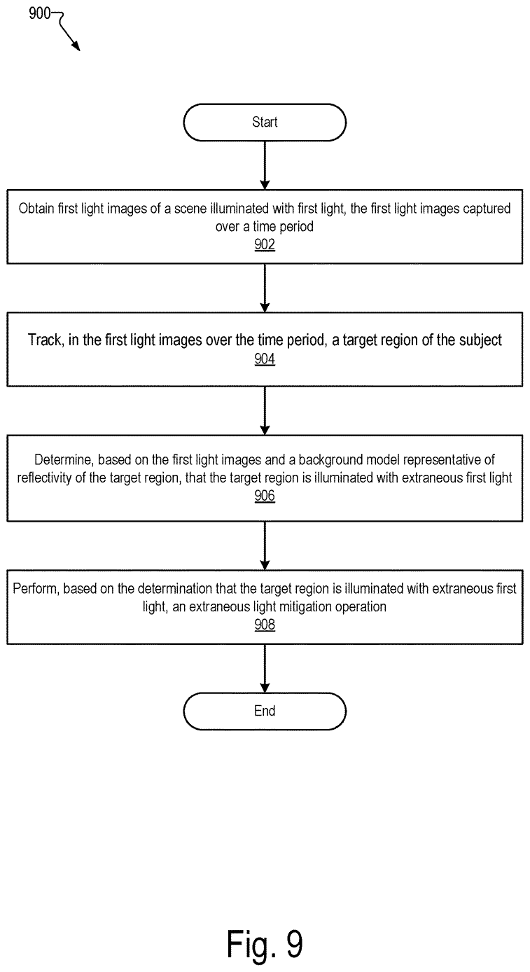

FIG. 9 shows an illustrative method 900 of detecting and mitigating extraneous light. While FIG. 9 shows operations according to one embodiment, other embodiments may omit, add to, reorder, and/or modify any of the operations shown in FIG. 9. One or more of the operations shown in FIG. 9 may be performed by system 200, by any components included therein, and/or by any implementation thereof.

Operations of method 900 may be performed in any suitable way, including any way described herein.

At operation 902, system 200 obtains first light images of a scene illuminated with first light. The first light images are captured over a time period and depict a subject located at the scene. In some examples, signal levels of the first light images are adjusted to compensate for the uneven distribution of first light. In some examples, the first light images are visible light images of the scene illuminated with visible light (e.g., blue light).

At operation 904, system 200 tracks, in the first light images over the time period, a target region of the subject.

At operation 906, system 200 determines, based on the first light images and a background model representative of reflectivity of the target region, that the target region is illuminated with extraneous first light.

At operation 908, system 200 performs, based on the determination that the target region is illuminated with extraneous first light, an extraneous light mitigation operation.

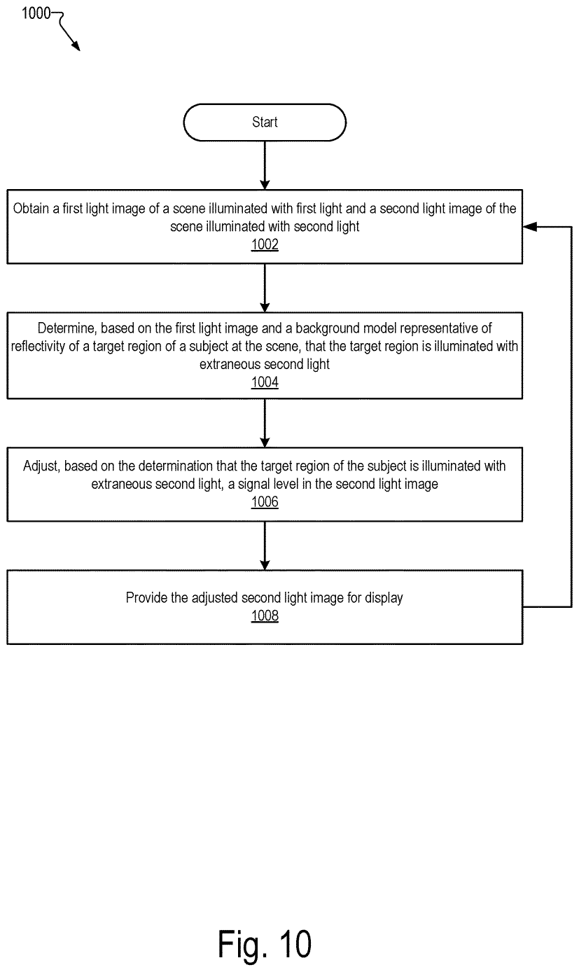

FIG. 10 shows an illustrative method 1000 of detecting and mitigating extraneous light. While FIG. 10 shows operations according to one embodiment, other embodiments may omit, add to, reorder, and/or modify any of the operations shown in FIG. 10. One or more of the operations shown in FIG. 10 may be performed by system 200, by any components included therein, and/or by any implementation thereof.

Operations of method 1000 may be performed in any suitable way, including any way described herein.

At operation 1002, system 200 obtains a first light image of a scene illuminated with first light and a second light image of the scene illuminated with second light. In some examples, the first light image and the second light image were captured at substantially the same time so that they represent substantially the same state of the scene. In some examples, signal levels of the first light image and/or the second light image are adjusted to compensate for the uneven distribution of first light and second light. In some examples, the first light image is a visible light image of the scene illuminated with visible light and the second light image is a fluorescence image of the scene illuminated with fluorescence excitation light.

At operation 1004, system 200 determines, based on the first light image and a background model representative of reflectivity (of first light) of a target region of a subject at the scene, that the target region is illuminated with extraneous second light.

At operation 1006, system 200 adjusts, based on the determination that the target region is illuminated with extraneous second light, a signal level in the second light image corresponding to the target region.

At operation 1008, system 200 provides the adjusted second light image for display by a display device. Processing then returns to operation 1002 to repeat the process with the next or a subsequently-acquired first light image and second light image.

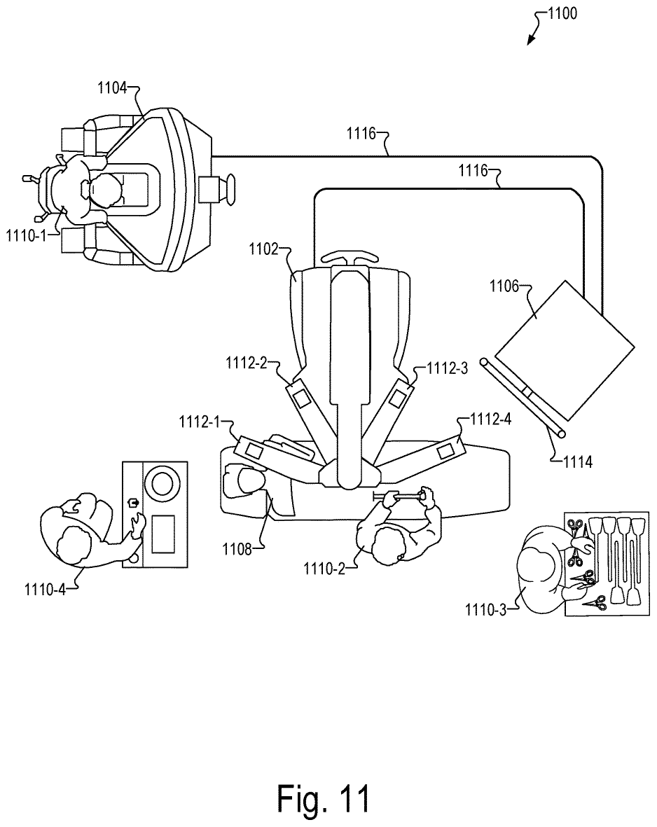

The systems and methods described herein may be used in conjunction with a computer-assisted surgical system. FIG. 11 shows an illustrative computer-assisted surgical system 1100 (“surgical system 1100”) that may be used in conjunction with the systems and methods described herein. As described herein, system 200 may be implemented by surgical system 1100, connected to surgical system 1100, and/or otherwise used in conjunction with surgical system 1100.

As shown, surgical system 1100 includes a manipulating system 1102, a user control system 1104, and an auxiliary system 1106 communicatively coupled one to another. Surgical system 1100 may be utilized by a surgical team to perform a computer-assisted surgical procedure on a subject 1108. As shown, the surgical team may include a surgeon 1110-1, an assistant 1110-2, a nurse 1110-3, and an anesthesiologist 1110-4, all of whom may be collectively referred to as “surgical team members 1110.” Additional or alternative surgical team members may be present during a surgical session as may serve a particular implementation.

While FIG. 11 illustrates an ongoing minimally invasive surgical procedure, it will be understood that surgical system 1100 may similarly be used to perform open surgical procedures or other types of surgical procedures that may similarly benefit from the accuracy and convenience of surgical system 1100. Additionally, it will be understood that the surgical session throughout which surgical system 1100 may be employed may not only include an operative phase of a surgical procedure, as is illustrated in FIG. 11, but may also include preoperative, postoperative, and/or other suitable phases of the surgical procedure. A surgical procedure may include any procedure in which manual and/or instrumental techniques are used on a subject to investigate, diagnose, and/or treat a physical condition of the subject. Additionally, a surgical procedure may include any non-clinical procedure, e.g., a procedure that is not performed on a live subject, such as a calibration or testing procedure, a training procedure, and an experimental or research procedure.

As shown in FIG. 11, manipulating system 1102 includes a plurality of manipulator arms 1112 (e.g., manipulator arms 1112-1 through 1112-4) to which a plurality of surgical instruments may be coupled. Each surgical instrument may be implemented by any suitable surgical tool (e.g., a tool having tissue-interaction functions), medical tool, imaging device (e.g., an endoscope), sensing instrument (e.g., a force-sensing surgical instrument), diagnostic instrument, or the like that may be used for a computer-assisted surgical procedure on subject 1108 (e.g., by being at least partially inserted into subject 1108 and manipulated to perform a computer-assisted surgical procedure on subject 1108). While manipulating system 1102 is depicted and described herein as including four manipulator arms 1112, manipulating system 1102 may include only a single manipulator arm 1112 or any other number of manipulator arms as may serve a particular implementation.

Manipulator arms 1112 and/or surgical instruments attached to manipulator arms 1112 may include one or more displacement transducers, orientational sensors, and/or positional sensors used to generate raw (i.e., uncorrected) kinematics information. One or more components of surgical system 1100 may be configured to use the kinematics information to track (e.g., determine positions and orientations of) and/or control the surgical instruments.

User control system 1104 is configured to facilitate control by surgeon 1110-1 of manipulator arms 1112 and surgical instruments attached to manipulator arms 1112.

For example, surgeon 1110-1 may interact with user control system 1104 to remotely move or manipulate manipulator arms 1112 and the surgical instruments. To this end, user control system 1104 provides surgeon 1110-1 with images (e.g., high-definition 3D images, composite medical images, and/or fluorescence images) of a surgical area associated with subject 1108 as captured by an imaging system (e.g., imaging system 100). In certain examples, user control system 1104 includes a stereo viewer having two displays where stereoscopic images of a surgical area associated with subject 1108 and generated by a stereoscopic imaging system may be viewed by surgeon 1110-1. Surgeon 1110-1 may utilize the images to perform one or more procedures with one or more surgical instruments attached to manipulator arms 1112.

To facilitate control of surgical instruments, user control system 1104 includes a set of master controls. The master controls may be manipulated by surgeon 1110-1 to control movement of surgical instruments (e.g., by utilizing robotic and/or teleoperation technology). The master controls may be configured to detect a wide variety of hand, wrist, and finger movements by surgeon 1110-1. In this manner, surgeon 1110-1 may intuitively perform a procedure using one or more surgical instruments.

Auxiliary system 1106 includes one or more computing devices configured to perform primary processing operations of surgical system 1100. In such configurations, the one or more computing devices included in auxiliary system 1106 may control and/or coordinate operations performed by various other components (e.g., manipulating system 1102 and user control system 1104) of surgical system 1100. For example, a computing device included in user control system 1104 may transmit instructions to manipulating system 1102 by way of the one or more computing devices included in auxiliary system 1106. As another example, auxiliary system 1106 may receive, from manipulating system 1102, and process image data (e.g., fluorescence image data 218 and/or processed fluorescence image data 226) representative of images captured by an imaging device (e.g., imaging device 102) attached to one of manipulator arms 1112.

In some examples, auxiliary system 1106 is configured to present visual content to surgical team members 1110 who may not have access to the images provided to surgeon 1110-1 at user control system 1104. To this end, auxiliary system 1106 may include a display monitor 1114 configured to display one or more user interfaces, such as images (e.g., 2D images, composite medical images, and/or fluorescence images) of the surgical area, information associated with subject 1108 and/or the surgical procedure, and/or any other visual content as may serve a particular implementation. For example, display monitor 1114 may display images of the surgical area together with additional content (e.g., graphical content, contextual information, etc.) concurrently displayed with the images. In some embodiments, display monitor 1114 is implemented by a touchscreen display with which surgical team members 1110 may interact (e.g., by way of touch gestures) to provide user input to surgical system 1100.

Manipulating system 1102, user control system 1104, and auxiliary system 1106 may be communicatively coupled one to another in any suitable manner. For example, as shown in FIG. 11, manipulating system 1102, user control system 1104, and auxiliary system 1106 are communicatively coupled by way of control lines 1116, which may represent any wired or wireless communication link as may serve a particular implementation. To this end, manipulating system 1102, user control system 1104, and auxiliary system 1106 may each include one or more wired or wireless communication interfaces, such as one or more local area network interfaces, Wi-Fi network interfaces, cellular interfaces, etc.

The apparatuses, systems, and methods described herein have been described with reference to fluorescence. However, it will be appreciated that the systems and methods described herein are not limited to fluorescence but may be applied to any other type of luminescence, including but not limited to photoluminescence (e.g., phosphorescence, etc.), electroluminescence, chemiluminescence, mechanoluminescence, radioluminescence, and the like.

Moreover, the scene may be illuminated with NIR light for purposes other than fluorescence imaging, such as spatial frequency domain imaging (SFDI) and other optical imaging techniques. The systems and methods described herein may be used to detect extraneous NIR light at a target region of the scene and perform a mitigation operation, including adjusting signal levels of captured images based on the detected extraneous NIR light. Moreover, the apparatuses, systems, and methods described herein may be used to detect and mitigate any extraneous electromagnetic energy (such as ultraviolet light and infrared light) incident on a target region of a subject at a scene.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g., a hard disk, a floppy disk, magnetic tape, etc.), ferroelectric random-access memory (RAM), and an optical disc (e.g., a compact disc, a digital video disc, a Blu-ray disc, etc.). Exemplary volatile storage media include, but are not limited to, RAM (e.g., dynamic RAM).

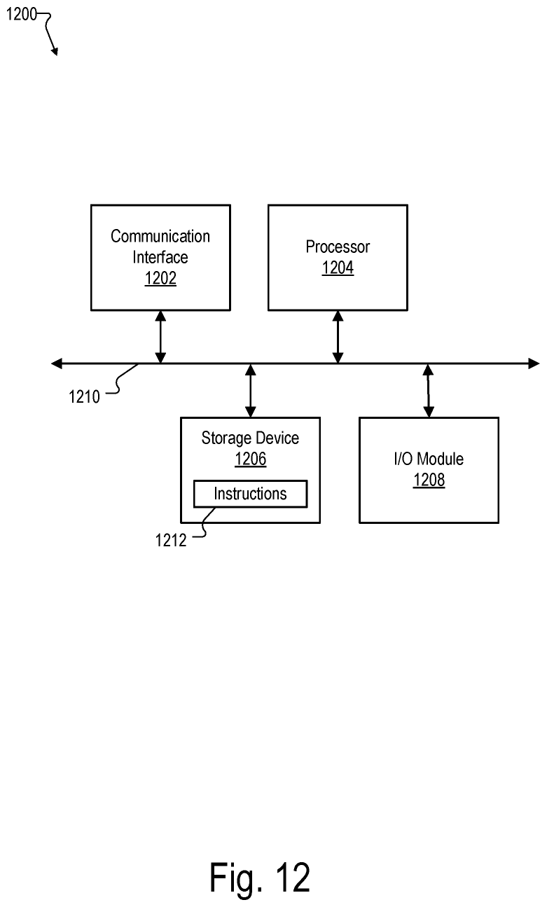

FIG. 12 shows a functional diagram of an illustrative computing device 1200 that may be specifically configured to perform one or more of the processes described herein. Any of the systems, units, computing devices, and/or other components described herein may be implemented by computing device 1200.

As shown in FIG. 12, computing device 1200 may include a communication interface 1202, a processor 1204, a storage device 1206, and an input/output (I/O) module 1208 communicatively connected one to another via a communication infrastructure 1210. While an exemplary computing device 1200 is shown in FIG. 12, the components illustrated in FIG. 12 are not intended to be limiting. Additional or alternative components may be used in other embodiments. Components of computing device 1200 shown in FIG. 12 will now be described in additional detail.

Communication interface 1202 may be configured to communicate with one or more computing devices. Examples of communication interface 1202 include, without limitation, a wired network interface (such as a network interface card), a wireless network interface (such as a wireless network interface card), a modem, an audio/video connection, and any other suitable interface.

Processor 1204 generally represents any type or form of processing unit capable of processing data and/or interpreting, executing, and/or directing execution of one or more of the instructions, processes, and/or operations described herein.

Processor 1204 may perform operations by executing computer-executable instructions 1212 (e.g., an application, software, code, and/or other executable data instance) stored in storage device 1206.

Storage device 1206 may include one or more data storage media, devices, or configurations and may employ any type, form, and combination of data storage media and/or device. For example, storage device 1206 may include, but is not limited to, any combination of the non-volatile media and/or volatile media described herein.

Electronic data, including data described herein, may be temporarily and/or permanently stored in storage device 1206. For example, data representative of computer-executable instructions 1212 configured to direct processor 1204 to perform any of the operations described herein may be stored within storage device 1206. In some examples, data may be arranged in one or more databases residing within storage device 1206.

I/O module 1208 may include one or more I/O modules configured to receive user input and provide user output. I/O module 1208 may include any hardware, firmware, software, or combination thereof supportive of input and output capabilities.

For example, I/O module 1208 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1208 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
a processor communicatively coupled to the memory and configured to execute the instructions to:
obtain first light images of a scene illuminated with first light in a first waveband and captured over a time period, the first light images depicting a subject located at the scene;
track, in the first light images over the time period, pixel values of a target region of the subject; and
determine, based on a comparison of signal levels of pixels that depict the target region of the subject with a background model representative of reflectivity of the target region, whether the target region is illuminated with extraneous first light.

2. The system of claim 1, wherein:
the processor is further configured to execute the instructions to adjust, based on an incident first light model representative of a three-dimensional spatial distribution of first light from a first light source, the signal levels of the pixels that depict the target region in the first light images; and
the comparison is performed with the adjusted signal levels of the pixels that depict the target region.

3. (canceled)

4. The system of claim 1, wherein:
the scene is further illuminated with second light during the time period; and
the processor is further configured to execute the instructions to:
obtain second light images of the scene captured over the time period;
estimate, based on an incident second light model representative of a three-dimensional spatial distribution of second light from a second light source, an amount of second light incident on the target region; and
adjust signal levels of the second light images based on the estimated amount of second light incident on the target region.

5. The system of claim 1, wherein the processor is further configured to execute the instructions to determine, based on a determination that the target region is illuminated with extraneous first light, that the target region is illuminated with extraneous second light.

6. The system of claim 5, wherein the processor is further configured to execute the instructions to:
estimate, based on the background model and the first light images, an amount of extraneous second light incident on the target region; and
adjust the signal levels of the second light images further based on the estimated amount of extraneous second light incident on the target region.

7-10. (canceled)

11. The system of claim 1, wherein the processor is further configured to execute the instructions to perform an extraneous light mitigation operation based on a determination that the target region is illuminated with extraneous first light.

12-13. (canceled)

14. The system of claim 1, wherein the processor is further configured to execute the instructions to generate, over the time period, the background model based on the signal levels of the pixels that depict the target region.

15. The system of claim 14, wherein the background model is generated further based on signal levels of pixels that depict one or more additional target regions in additional first light images captured over one or more prior time periods.

16. The system of claim 1, wherein:
the subject comprises tissue; and
the processor is further configured to execute the instructions to:
determine a type of the tissue at the target region; and
select the background model based on the determined type of the tissue at the target region.

17. The system of claim 1, wherein the processor is configured to execute the instructions to determine that the target region is illuminated with extraneous first light based on a determination that the signal levels of the pixels that depict the target region exceed a threshold signal level of the background model for a threshold number of frames or for a threshold period of time.

18. The system of claim 1, wherein the processor is configured to execute the instructions to determine that the target region is illuminated with extraneous first light based on a determination that the signal levels of the pixels that depict the target region exceed a threshold signal level of the background model by at least a threshold amount.

19. The system of claim 1, wherein the processor is further configured to execute the instructions to select the target region based on user input.

20. The system of claim 1, wherein the processor is further configured to execute the instructions to automatically select the target region based on image segmentation.

21. The system of claim 1, wherein the pixels that depict the target region comprise a subset of all pixels of each frame of the first light images.

22. The system of claim 1, wherein the processor is further configured to execute the instructions to determine, based on the signal levels of the pixels that depict the target region in the first light images, a reflectivity of the target region.

23. A method comprising:
obtaining first light images of a scene illuminated with first light in a first waveband and captured over a time period, the first light images depicting a subject located at the scene;
tracking, in the first light images over the time period, pixel values of a target region of the subject; and
determining, based on a comparison of signal levels of pixels that depict the target region with a background model representative of reflectivity of the target region, whether the target region is illuminated with extraneous first light.

24. The method of claim 23, further comprising:
adjusting, based on an incident first light model representative of a three-dimensional spatial distribution of first light from a first light source, the signal levels of the pixels that depict the target region in the first light images;
wherein the comparison is performed with the adjusted signal levels of the pixels that depict the target region.

25. (canceled)

26. The method of claim 23, wherein:
the scene is further illuminated with second light during the time period; and
the method further comprises:
obtaining second light images of the scene captured over the time period;
estimating, based on an incident second light model representative of a three-dimensional spatial distribution of second light from a second light source, an amount of second light incident on the target region; and
adjusting signal levels of the second light images based on the estimated amount of second light incident on the target region.

27. The method of claim 23, further comprising determining, based on a determination that the target region is illuminated with extraneous first light, that the target region is illuminated with extraneous second light.

28-52. (canceled)

53. A non-transitory computer-readable medium storing instructions that, when executed, direct at least one processor of a computing device to:
obtain first light images of a scene illuminated with first light in a first waveband and captured over a time period, the first light images depicting a subject located at the scene;
track, in the first light images over the time period, pixel values of a target region of the subject; and
determine, based on a comparison of signal levels of pixels that depict the target region of the subject with a background model representative of reflectivity of the target region, whether the target region is illuminated with extraneous first light.
